(19) United States
(12) Patent Application Publication
(10) Pub. No.: US 2025/0152652 A1
(43) Pub. Date: May 15, 2025

(54) PHARMACEUTICAL COMPOSITION, PHYTOMEDICINE, KIT, PROCESS FOR PREPARING A PHARMACEUTICAL COMPOSITION, AND, USES OF ESSENTIAL OIL AND/OR A PHARMACEUTICAL COMPOSITION

(71) Applicant: BIOTEC TO LTDA., Gurupi (BR)

(72) Inventor: Robson Ruiz Olivoto, Gurupi (BR)

(21) Appl. No.: 18/963,716

(22) Filed: Nov. 28, 2024

(30) Foreign Application Priority Data
Nov. 9, 2023 (BR) 1020230250190

(51) Int. Cl. A61K 36/61; A61P 3/10

(52) U.S. Cl. CPC A61K 36/61; A61P 3/10; A61K 2236/331

(57) ABSTRACT

The present invention relates generally to a pharmaceutical composition comprising the essential oil of Campomanesia pubescens. The invention also provides a phytomedicine, a kit, a process for preparing said composition and uses for the oil and/or the composition. The invention is an innovative therapeutic alternative in the treatment and/or prevention of diabetes, based on the activity of controlling glycemic rate in an individual in need thereof.

### FIELD OF INVENTION

The present invention describes, in general terms, a pharmaceutical composition comprising the essential oil of Campomanesia pubescens. The invention also provides a phytomedicine, a kit, a process for preparing said composition and the uses of the oil and/or the composition in the control of glycemic rate and in the treatment and/or prevention of diabetes.

### BACKGROUND OF THE INVENTION

As described in the literature, diabetes mellitus is one of the most common pathologies among the Brazilian and world population. The treatment can be carried out in two ways, non-pharmacological and pharmacological. Non-pharmacological procedures are carried out through diet and exercise, while pharmacological procedures involve the use of exogenous insulin or another drug with a similar action.

According to the International Diabetes Federation, in its 2019 IDF IDABETES ATLAS, around 463 million people worldwide are diagnosed with diabetes of some kind. Added to this number is the prospect that 237 million are pre-diabetic, without a pathological diagnosis, so we will reach the year 2045, with approximately 700 million people classified as diabetic.

Today, it is estimated that the cost of treating people with diabetes each year is around US$3,117.00, meaning that by 2045 the annual cost of treating this condition will reach approximately US$2,181,900,000,000.00.

In Brazil, according to information from the BRAZILIAN DIABETES SOCIETY, published in the 2019/2020 GUIDELINES, the number of diabetics today is approximately 12.5 million people, with a cost, calculated on the basis of the IDF estimate, of US$38,962,500,000.00. The number of pre-diabetics amounts to approximately 7.8 million Brazilians, which would raise this number to 20.3 million Brazilians by the year 2045, increasing the cost of treatment to US$63,275,100,000.00 (in current values).

During 2021, an exploratory Desk survey was carried out, focusing on gathering information that would lead to a more in-depth investigation into the pain or possible pain that affected or affects diabetic people. An ethnographic survey was organized based on this body of information.

The ethnographic survey questionnaires were answered by 1,817 people. The research focused on identifying the age group, gender, whether or not they were insulin-dependent, and if they were insulin-dependent, the source from which they acquired the drug, and finally what the pains of diabetic people were.

In terms of age group, the majority of diabetics who answered the questionnaires were under 50, male and non-insulin dependent diabetics. By analyzing the age group (longevity), gender (less concern with basic dietary care), it can be assumed that a large percentage of this non-insulin dependent group will become insulin dependent.

A small percentage of insulin-dependent diabetics buy their insulin from their own resources; the majority get their medication from public health clinics. In terms of the person's pain, there is the best information, with over 85% of respondents indicating that the side effects of insulin and the application process (syringe) are the factors that cause the most discomfort and pain.

There is therefore a need in the state of the art to develop a low-cost agent capable of controlling glycemic rate in diagnosed diabetics and pre-diabetics, with the potential to replace the use of insulin. In this way, it would be possible to reduce the number of diabetic people by treating pre-diabetics.

For this, it is important to have effective and safe medicines, mainly of natural origin and with low toxicity. These natural products must make up pharmaceutical formulations for use by appropriate routes.

Campomanesia is a gender found from northern Argentina to Trinidad, and from the coast of Brazil to the Andes, Peru, Ecuador and Colombia. It has 36 known species, 31 of which are from the Brazilian flora. It is a clearly defined family, a monophyletic gender, with a developed hypocotyl and small cotyledons, trees or shrubs, and is distinguished from other genres by the way it develops its fruit and seeds, with the ovary having 4 to 18 locules, serving as a false envelope in seeds that have a membranous testa.

They have a woody habit, rarely shrubs, predominantly trees, leaves with translucent glands that produce terpenes and other aromatic substances, devoid of stipules, opposite phyllotaxis, predominantly white flowers, dialypetalous, multistaminate, inferior ovary, frequent bacaceous fruit and persistent calyx in most species.

Morphologically, they can appear as trees of around 15 meters, characteristic of tropical and subtropical forests, to shrubs of 1 meter found in cerrado grasslands. When ripe, the fruit has a hard, thick, exfoliating rind with fibers or thin papyraceous blades. The inflorescence is uniflorous or dicentric, usually with three flowers and solitary peduncles. The bracteoles are usually linear to elliptical and generally deciduous at anthesis, the ripe fruits are yellowish-green, yellow, orange or black, globose, about 1 to 3 cm long, very aromatic and palatable.

Studies carried out on extracts of plants from the Campomanesia family suggest their therapeutic effects. However, few studies have been carried out using the Campomanesia pubescens species used in the present invention.

In the search for the state of the art, the following documents dealing with the subject were found.

Vinagre, A. S. et al. studied the species Campomanesia xanthocarpa (leaf decoction). Treatment of diabetic rats with the decoction lowered blood glucose levels, inhibited the degradation of liver glycogen and prevented possible histopathological amendments in the pancreas and kidney. The results suggest that treatment with the decoction of C. xanthocarpa leaves (20 g/L) may be useful for managing diabetes mellitus but emphasize that pharmacological and toxicological studies are still needed.

Cardozo, C. M. L. et al. present a review of the therapeutic indications for species of the Campomanesia family. The authors report that the varieties contain different bioactive compounds with high antioxidant capacity and no clinical or reproductive toxicity effects, listing effects such as weight reduction and glycemic control of this family of plants. Specifically, about Campomanesia pubescens, it is stated that it exhibited high antioxidant activity along with antitumor activity in the in vitro assay of human tumor cell lines, reduced the number of monocytes (anti-inflammatory potential) and decreased plasma levels of liver enzymes (ALT and AST) without renal toxicity.

From the literature search, no documents were found anticipating or suggesting the teachings of the present invention, i.e. a pharmaceutical composition comprising the essential oil of Campomanesia pubescens with a specific constitution and concentration, as described below, nor even a phytomedicine, kit, preparation process and uses such as those presented here.

The pharmaceutical composition of the invention can be ingested in capsule form, eliminating discomfort for the insulin-dependent patient, since it eliminates the need for subcutaneous application and the costs of buying and disposing of syringes.

As it is a highly diffusible oil, its active ingredient has lipid characteristics and therefore a high diffusion coefficient, so its action does not require plasma or cellular transporters. What's more, the oil has no intoxicating effect, thus eliminating another disadvantage of exogenous insulin.

The action of the oil, as indicated by the results of the present invention, is to promote the absorption of plasma glucose by the tissues, controlling the glycemic rate. In this sense, it can be very effective in reducing blood sugar levels in anyone, whether diabetic or pre-diabetic.

In the case of diabetics, it can act as a controller of the pathology's evolutionary process, whether from a mild to high insulin-dependent diabetic state, or from a non-insulin-dependent to an insulin-dependent state. In the case of pre-diabetics, it can act by controlling the progression to the pathological diabetic state.

Thus, it is an objective of the present invention to provide a product of plant origin, from the essential oil of a plant, in particular Campomanesia pubescens, as an innovative therapeutic alternative capable of controlling the glycemic rate in an individual in need thereof, treating and/or preventing diabetes, with the potential to replace the use of exogenous insulin at a low cost, both in production and marketing.

### SUMMARY OF THE INVENTION

The present invention, in its most general aspect, describes a pharmaceutical composition comprising the essential oil of Campomanesia pubescens.

In one aspect, the invention provides a phytomedicine for controlling glycemic rate comprising capsules containing from about 90 μl to about 500 μl of the essential oil obtained by extracting the leaves of plants of the Campomanesia pubescens species.

In another aspect, the invention relates to a kit comprising (a) the essential oil of Campomanesia pubescens in a dosage ranging from about 90 μl to about 500 μl; and (b) instructions for use.

In yet another aspect, the invention provides a process for preparing said pharmaceutical composition comprising the following steps:
(a) collect the leaves of Campomanesia pubescens in the period before the fruit ripens;
(b) clean with plenty of water;
(c) extracting the essential oil by hydrodistillation and/or steam drag; and
(d) after extracting the oil, keep in amber glass to preserve its composition until final encapsulation.

In an additional aspect, the invention also relates to the use of Campomanesia pubescens essential oil for the manufacture of a pharmaceutical composition for controlling glycemic rate in an individual in need thereof.

In a further aspect, the invention relates to the use of said pharmaceutical composition for the manufacture of a phytomedicine for controlling glycemic rate in an individual in need thereof.

In yet another additional aspect, the invention relates to the use of Campomanesia pubescens essential oil or said pharmaceutical composition for the manufacture of a drug to treat and/or prevent diabetes.

### BRIEF DESCRIPTION OF THE FIGURES

The detailed description below refers to the attached figures:

FIG. 1: Experimental procedures—Extraction of essential oils.

FIG. 2: Dose-response curve and antioxidant capacity. Diagram illustrating the hydrodistillation process used to obtain oil from Campomanesia pubescens leaves.

FIG. 3: Concentration of metabolites and enzymes after 30 days of using CP oil.

FIG. 4: Agarose gel with the band corresponding to GLUT-4 mRNA.

The detailed description of the invention described below refers to the attached figures representing the embodiments of the present invention.

### DETAILED DESCRIPTION OF THE INVENTION

The detailed description of the present invention can be used to its fullest extent. The specific embodiments described below should be interpreted as merely illustrative and not as limiting the rest of the description in any way.

Unless otherwise defined, all technical and scientific terms used here have the same meaning as the invention. The terminology used in the description of the invention is intended to describe particular executions and is not intended to limit the scope of the teachings.

Unless otherwise indicated, all numbers expressing quantities, percentages and proportions, and other numerical values used in the descriptive report and in the claims, are to be understood as being modified, in all cases, by the term “about”. Therefore, unless otherwise stated, the numerical parameters shown in the descriptive report and in the claims are approximations that may vary depending on the properties to be obtained.

All patent and non-patent references cited in this application are hereby incorporated by reference in their entirety.

The present invention, in its most general aspect, describes a pharmaceutical composition comprising the essential oil of Campomanesia pubescens.

In the context of this patent application, essential oil should be understood as a concentrated mixture characterized by an intense odor that is liquid at room temperature and comprises several chemical components with distinct natural volatile and lipophilic chemical structures, being obtained from a specific process for obtaining essential oil from aromatic plants. However, it should also be understood that this is not simply a matter of extracting the essential oil from nature, as there are numerous possibilities for variations in the composition of the essential oil, depending on the process used to obtain the essential oil and the region from which the plant species is obtained.

The concentration of the essential oil in the composition can vary and will depend on a variety of factors, including the condition to be treated, the desired effect, the ability and speed to reach its intended target and other factors within the knowledge of the patient and the doctor.

In one embodiment, the essential oil of Campomanesia pubescens is in a concentration of around 90 μl to around 500 μl.

In another embodiment, the pharmaceutical composition comprises a pharmaceutically acceptable excipient.

For the purposes of the present invention, the term “pharmaceutically acceptable excipient” means that it is suitable for oral use without undue toxicity, incompatibility, instability, irritation or allergic reaction, or the like.

Pharmaceutically acceptable excipients are selected according to the final presentation of the formulation. Pharmaceutically acceptable excipients show no toxicity to the recipient organism at the dosages and concentrations used where they are static. References on pharmaceutically acceptable excipients are the publication “Remington's Pharmaceutical Sciences”, 18^{th} edition, (1990), Mack Publishing Co., USA, and the publication “Handbook of Pharmaceutical Additives” by Michael and Irene Ash, 1995, Gower (England).

Non-limiting examples of excipients suitable for the invention include surfactants such as tweens, spans and myrjs; gelling agents such as carboxymethyl, hydroxyethyl, hydroxypropyl celluloses; non-ionic emulsifiers such as monoglycerides, fatty acid esters, monoleates, polysorbates; immobilizing agents such as cyclodextrins: alpha and beta hydroxypropyl, alginates and gelatins; film-forming or filming agents, such as, polyvinyl alcohol and cetostearyl alcohol; epithelizing agents, such as, allantoin and cepalin; dispersing agents, such as sorbitol 70%, polyethylene glycol, stearates; and preserving and stabilizing agents, such as parabens and imidasolinidyl urea, used alone or in combination with each other; carrier agents such as coconut oil.

In one embodiment, pharmaceutically acceptable excipients can be selected from the classes consisting of diluents, carriers, protectants, surfactants, dispersing agents, immobilizing agents, film-forming or film-forming agents, healing agents, tissue regenerating agents, stabilizing agents, gelling agents, epithelializing agents, non-ionic emulsifiers, preserving agents and vehicles.

In the present invention, the pharmaceutically acceptable excipient is in a concentration of about 100 μl to about 600 μl.

In one embodiment, the pharmaceutical composition containing the essential oil of Campomanesia pubescens of the present invention is administered orally.

In other embodiment, the pharmaceutical composition is in the form of capsules, tablets, suspensions and the like.

In a preferred embodiment, this composition is in capsule form.

The encapsulation technique has been known for many years. Normally, these pharmaceutical capsules are made of gelatine or some modification of it, which is essentially manufactured in two different forms, namely the so-called hard gelatine capsule and the soft gelatine capsule.

Soft gelatine capsules generally comprise an outer shell made primarily of gelatine, a plasticizer and water, and a filler contained within the shell. The filler can be selected from any of a variety of substances that are compatible with the gelatine shell. Soft gelatine capsules are available in a wide variety of sizes and shapes, including round shapes, oval shapes, oblong shapes, tubular shapes and other types of special shapes, such as stars. The finished capsules can be produced in a variety of colors. In addition, opacifiers can be added to the shell.

It is also known that conventional soft gelatine capsules are the preferred form of administration for medicines and similar products, especially liquids, pastes, solids dispersed in liquids or dry solids. Soft gelatine capsules also have advantages for substances that need total isolation from air and light, as the gelatine is completely sealed around the contents.

Hard gelatine capsules are also known in the technique, and are usually made up of two distinct parts, called the “lid” and the “body”, which fit together to form the complete capsule. The lid and body are manufactured using the same process, which consists of immersing the end of a mandrel whose shape corresponds to the internal volume of the lid or body in a gelatine solution, then removing the mandrel from the solution and allowing the layer of gelatine thus deposited to dry, which is then removed like the finger of a glove.

Specifically, the pharmaceutical composition of the present invention has a lipid characteristic with a high diffusion coefficient.

In a preferred embodiment, this composition is in the form of a soft gelatine capsule.

According to the invention, the chemical constituents of Campomanesia pubescens essential oil can be extracted from the species or synthesized and used to formulate medicines for controlling glycemic rate.

In one aspect, the invention provides a phytomedicine for controlling glycemic rate comprising capsules containing from about 90 μl to about 500 μl of the essential oil obtained by extracting the leaves of plants of the Campomanesia pubescens species.

This essential oil can be used in oral herbal medicine formulations in the form of tablets, capsules, suspensions and the like, all of which are used for controlling glycemic rate.

In one embodiment, the phytomedicine is in capsule form.

In another aspect, the invention relates to a kit for controlling glycemic rate comprising (a) the essential oil of Campomanesia pubescens in a dosage ranging from about 90 μl to about 500 μl; and (b) instructions for use.

The kit can also include a container. Suitable containers include, for example, bottles, flasks (e.g. double chamber flask) and test tubes. In some cases, the container is a bottle. The container can be formed from a variety of materials such as glass or plastic. The container retains the composition.

The kit may additionally comprise a label or package leaflet, which is on or associated with the container and may indicate directions for reconstitution and/or use of the composition. The label or package leaflet may additionally indicate that the composition is useful or intended for oral administration or other modes of administration for controlling glycemic rate in an individual. The container containing the composition can be a single-use vial or a multi-use vial, which allows repeated administrations of the reconstituted composition. The kit can also include a second container containing a suitable diluent. The kit may additionally include other materials that are desirable from a commercial, therapeutic, and user point of view, including other buffers, diluents, filters, needles, syringes, and leaflets with instructions for use.

Such a kit can additionally include, if desired, one or more of the various conventional pharmaceutical components, such as, for example, containers with one or more pharmaceutically acceptable excipients, additional containers, etc., as will be readily apparent to those skilled in the art. Printed instructions, such as leaflets or labels, indicating the quantities of the components to be administered, guidelines for administration and/or guidelines for mixing the components can also be included in the kit.

In yet another aspect, the present invention provides a process for preparing said pharmaceutical composition comprising the following steps:
(a) collect the leaves of Campomanesia pubescens in the period before the fruit ripens;
(b) clean with plenty of water;
(c) extracting the essential oil by hydrodistillation and/or steam drag; and
(d) after extracting the oil, keep in amber glass to preserve its composition until final encapsulation.

In a further aspect, the invention also relates to the use of the pharmaceutical composition as defined above for the manufacture of a drug for controlling glycemic rate.

In a further aspect, the invention relates to the use of said pharmaceutical composition for the manufacture of a phytomedicine for controlling glycemic rate in an individual in need thereof.

In yet another additional aspect, the invention relates to the use of Campomanesia pubescens essential oil or said pharmaceutical composition for the manufacture of a drug to treat and/or prevent diabetes.

Although various forms of realization embodying the teachings of the present invention have been shown and described in detail here, any person skilled in the art will readily be able to envision many other varied forms of embodiments that may yet embody these teachings. Any equivalent aspects are intended to be within the scope of this invention.

The present invention will now be described by means of examples, which are intended to be merely illustrative of the present invention and are in no way limiting as to the scope and breadth of the present invention.

### EXAMPLES

### Materials and Methods

### Example 1: Essential Oil Extraction

The essential oils from the leaves were extracted according to the method described in the Brazilian Pharmacopoeia using a modified Clevenger extractor from a mixture of 3.673 g of the plant's fresh leaves with 200 mL of distilled water, previously crushed in a blender for 15 minutes. The crushed material was then transferred to a 250 mL round-bottomed flask and placed in the heating mantle that was attached to the Clevenger apparatus and kept at a temperature of 100° C. After 2 hours of extraction, the water/oil mixture was dried with anhydrous sodium sulphate.

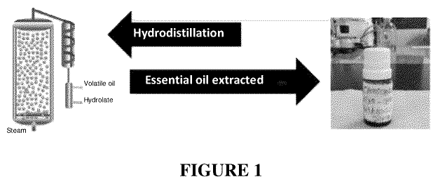

The leaves were collected from native Campomanesia pubescens plants, belonging to the Myrtaceae family, also known as guavirova, gabiroba, guavirá, among other names. The fruit was collected between 15 and 10 days before it ripened, right at the end of the loss of the peduncle pubescens. The leaves were processed, crushed and the oil was extracted using the hydrodistillation method (as described in the previous paragraph). The oil was kept in amber bottles, isolated from light and kept in a cool, ventilated environment until it was administered (FIG. 1).

### Example 2: Analysis of Essential Oil Constituents

The analyses were carried out using a Varian model GC-3900 gas chromatograph coupled to a Varian model Saturn 2100 T/MS/MS mass spectrometer, equipped with a Combi Pal automatic injector and an ion trap mass analyzer. The Saturn GC/MS 5.52 software and the NIST 2.0 library were used for data acquisition.

For chromatographic separation, a VF-5 ms Factor Four fused silica capillary column was used (with a stationary phase of polydimethylsiloxane with 5% phenyl, 30 m long, 0.25 mm internal diameter and 0.25 μm thickness of the stationary phase film).

The carrier gas used was helium with a purity content of 99.999% at a constant flow rate of 1.0 mL^{min-1}. The injector temperature was 250° C. and injection was carried out as follows: splitless for 4 min, followed by a split ratio of 50:1 for 15 min and 20:1 for the rest of the run. The conditions for the column oven were as follows: initial temperature at 80° C. (4 min); 80-215° C. (15° C.^{min-1}, held for 0.5 min); 215-230° C. (2° C.^{min-1}, with 3 min isotherm); 230-260° C. (5° C.^{min-1}, with 2 min isotherm).

The detector temperatures selected were 200° C. in the ion trap, 50° C. in the manifold and 250° C. in the transfer line. Ion acquisition was carried out in linear scan analysis mode and was performed in the 60-430 m/z range. The ionization was by electron impact at 70 eV, the scan time was 0.5 s/scan and 65000 ion counts. The emission current of the ionization filament was 70 μA and the voltage of the electro-multiplier was 200 V.

TABLE 1
Identification of essential oil constituents: Retention
indices were determined based on data from Adams, 1995.

| Order | Composition | IR | % |
|---|---|---|---|
| 1. | a-Felandrena | 1005 | 0.3 |
| 2. | d-3-Carena | 1011 | 0.2 |
| 3. | a-tempinene | 1019 | 0.3 |
| 4. | trans-pinocarveol | 1133 | 15.7 |
| 5. | pinocarvana | 1156 | 3.7 |
| 6. | myrternal | 1189 | 27 |
| 7. | myrternol | 1192 | 24.7 |
| 8. | d-elernene | 1266 | 0.5 |
| 9. | a-capaeno | 1359 | 0.2 |
| 10. | b-burbanena | 1359 | 0.6 |
| 11. | b-elemene | 1393 | 0.5 |
| 12. | iso-caryophyllene | 1412 | 0.3 |
| 13. | a-muralene | 1494 | 0.5 |
| 14. | d-cadinene | 1505 | 0.4 |
| 15. | selene-3,7(11)-diene | 1547 | 0.8 |
| 16. | spathulenol | 1572 | 5.4 |
| 17. | caryophyllene oxide | 1576 | 5 |
| 18. | a-Acorenol | 1629 | 0.6 |
| 19. | ala-aramadendrene oxide | 1633 | 7.5 |
| 20. | y-Eudesmol | 1637 | 0.4 |
| 21. | epl-a-cadinol | 1642 | 2 |
| 22. | a-Muralol | 1645 | 0.5 |
| 23. | a-cadirol | 1651 | 1.2 |

| | |
|---|---|
| Monoterpellic hydrocarbons | 54.1 |
| Oxygenated menoterpenes | 8.2 |
| Sesquiterpene hydrocarborides | 19.7 |
| Oxygenated sesquilterpenes | 16.3 |
| Total identified | 95.30% |

### Example 3: Preparation of the Pharmaceutical Composition Comprising Campomanesia pubescens Essential Oil

The preparation of the pharmaceutical composition comprises the following steps: 1) Collection of the plant material; Campomanesia pubescens leaves; 2) Extraction of the essential oil from the fresh plant material by the hydrodistillation method in a Clevenger-type apparatus for two hours or by steam drag in suitable equipment; 3) Storage of the extracted essential oil in amber glassware, isolated from light and kept in a cool, airy environment; 4) Qualitative and quantitative analysis of the essential oil by gas chromatography coupled with mass spectrometry; 5) Addition of a pharmaceutically acceptable excipient; 6) Encapsulation; 7) Packaging;

The encapsulation procedure was carried out using a base plate with holes for depositing the capsular halves for packaging. The procedure was complemented by the upper plate, with holes corresponding to the size of the capsules and a cavity where the oily volume was added.

To carry out the packaging, a centrifuge was used to agitate and homogenize the solution; micropipettes to measure the quantities of materials; a pharmaceutically acceptable excipient, such as a carrier agent such as coconut oil, to act as an encapsulating substance and fill the capsule; and the vegetable essential oil extracted from the leaves of Campomanesia pubescens (CP).

The procedures were the halves of the empty gelatine capsules (commercial standard) were added to the holes in the base plates; the cavity of the base plate was filled with 120 microliters of Campomanesia pubescens oil plus 180 microliters of coconut oil; the filling procedures were carried out using micropipettes; after filling the base plate, the top plate was positioned on top and the holes were aligned to close the capsules; after light pressure to fill the entire capsule with the oil, the parts of the plate were separated and, before storage, the capsules were dried in an oven in order to remove any moisture that may exist and/or amend the constitution of the formulation. As required by the pharmacopoeia, the capsules were stored in amber glass, protected from light and kept refrigerated.

The control of the glycemic rate was analyzed according to the treatment protocol below. The pharmaceutical composition comprising Campomanesia pubescens essential oil is able to control the glycemic rate in an individual in need thereof.

### Example 4: Sample Groups and Experimental Protocols

The sample, to determine the most efficient volume for controlling glycemic rate, consisted of male wistar norvegicus rats with an average weight of 300 grams. The animals were divided into control and experimental groups. The experimental group was subdivided into 5 groups, while the control group was divided into control (+) and control (−). The control group (−) and the experimental groups were treated with alloxan (120 mg/kg) (Dias da Silva, et al, 2014; Ferreira, 2017) to induce the diabetic state. The experimental groups were treated with essential oil extracted from Campomanesia pubescens (CP) in the following volumes: 30, 60, 90, 120 and 150 μl. The antioxidant potential (DPPH) was determined using the method described by Brand-Williams et al. (1995). The possible renal and hepatic toxicity of the oil at volumes of 90, 120 and 150 μl was analyzed. Possible toxicity was determined based on urea and creatinine levels (kidney function) and the enzymes alanine aminotransferase and aspartate aminotransferase (liver function).

Plasma biochemical parameters were analyzed using blood tests. The identification of the possible route of action of the oil was initially determined using the HPLC method followed by agarose gel (PCR) with the selected volume. The material for analysis was collected from diabetic animals with and without treatment and normal animals. The data was analyzed using Student's t-test and/or ANOVA (one-way or two-way). When ANOVA revealed a significant difference, the Tukey test was applied. The data was analyzed, and the figures plotted using GraphPad Prism System software (San Diego, CA, U.S.A.).

The procedures began with inhalation anesthesia, after which the tails were immersed in water at a temperature higher than room temperature to cause vasodilation of the caudal vein. Blood was collected by direct puncture of the caudal vein. This first volume was analyzed to determine the baseline glycemic rate, before treatment, i.e. time 0 (zero) or the standard for determining the diabetic glycemic rate. The experimental animals were then treated with different volumes of oil extracted from Campomanesia pubescens leaves. The treatment was carried out using the gavage method, avoiding errors in the administration of the volume. After 30 minutes of administration, a new blood sample was taken to analyze the glycemic rate, and the same procedure was carried out 60 minutes after the treatment.

### Results

### Example 5: Controlling of Glycemic Rate

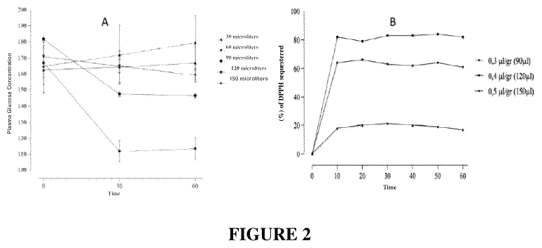

The results indicate that the oil is able to control the plasma glycemic rate in diabetic animals. The results presented by the dose-response curve protocol indicate that the 90 and 120 μl volumes were effective in reducing plasma glycemic rates; however, the 120 μl volume was able to normalize blood glucose levels in diabetic animals in less than 30 minutes, maintaining control up to 60 minutes (FIG. 2).

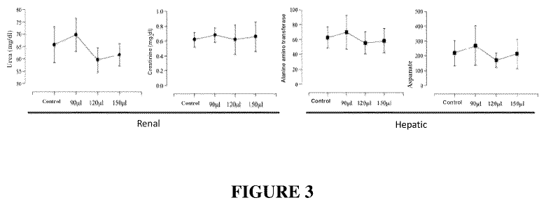

As observed in the dose response protocol, the antioxidant capacity of the oil is also associated with the 90 and 120 μl volumes, and again the 120 μl volume was the most efficient (FIG. 3).

Statistically, the volumes of 90, 120 and 150 μl did not promote an intoxicating response in renal and hepatic functions when compared to the control group. About biochemical parameters, the volume of 120 μl promoted an increase in the expression of the leukocyte family (immune response).

TABLE 2
Biochemical parameters analyzed in animals treated
with CP oil for 30 days at different doses.

| | | CP | CP | CP |
|---|---|---|---|---|
| Parameters | Control | (90 microliters) | (120 microliters) | (150 microliters) |
| Hematocrit | 43 ± 1.1 | 43 ± 1.5 | 44 ± 2.6 | 44 ± 2.4 |
| Leukocytes | 5872 ± 1222.1 | 6521 ± 1972.2 | 8061 ± 1549.0 | 6973 ± 1256.5 |
| Basophils | 0 ± 0.0 | 0 ± 0.0 | 0 ± 0.0 | 0 ± 0.0 |
| Eosinophils | 0.4 ± 0.9 | 0.2 ± 0.4 | 0.2 ± 0.4 | 0.2 ± 0.4 |
| Band cells | 0.6 ± 0.9 | 0.4 ± 0.5 | 0.8 ± 0.8 | 0.6 ± 0.3 |
| Segmented | 10.1 ± 2.9 | 10.4 ± 4.8 | 12.9 ± 6.0 | 12.8 ± 4.0 |
| Lymphocytes | 79 ± 6.2 | 81.6 ± 5.9 | 77.4 ± 5.5 | 78.1 ± 7.1 |
| Monocytes | 17.2 ± 0.2 | 7.2 ± 0.7 | 9.2 ± 1.8 | 8.2 ± 3.9 |

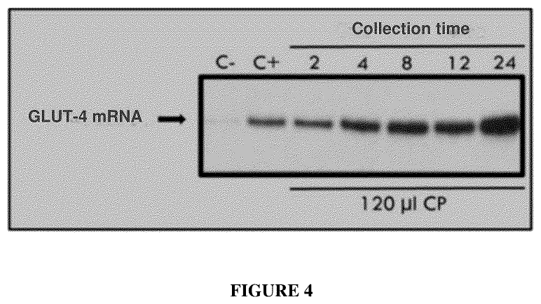

The gel data indicate that the action of the oil is directly on the release of the vesicle containing the GLUT-4 proteins (FIG. 4). Taken together, the data suggests that the oil acts in a similar way to endogenous insulin and has the property (active ingredient) to be an efficient and less expensive substitute for the treatment of diabetics.

### REFERENCES

VINAGRE, A. S. et al., Anti-diabetic effects of Campomanesia xanthocarpa (Berg) leaf decoction. Brazilian Journal of Pharmaceutical Sciences, Sao Paulo, v. 46, n. 2, p. 169-177, abr./jun. 2010. DOI 10.1590/S1984-82502010000200002.
Cardozo C. M. L. et al., Therapeutic Potential of Brazilian Cerrado Campomanesia Species on Metabolic Dysfunctions. Molecules. 2018 Sep. 13; 23(9):2336. doi: 10.3390/molecules23092336. PMID: 30216974; PMCID: PMC6225494.
R. P. Adams, Identification of Essential Oil Components by Gas Chromatography/Mass Spectroscopy. Allured Publ Corp, Carol Stream, IL (1995).
Silva et al., Evaluation of the antimicrobial activity of the crude ethanol extract, essential oil, and fractions from Campomanesia pubescens leaves, Research, Society and Development, v. 11, n. 5, e56911528622, 2022.
Cardoso, C. A. L. et al., Avaliagio da atividade antioxidante, toxicidade e composição química por CG-EM do extrato hexânico das folhas de Campomanesia pubescens, Rev. Ciênc. Farm. Básica Apl., v. 29, n. 3, p. 297-301, 2008.
J Kusari, et al., Analysis of the gene sequences of the insulin receptor and the insulin-sensitive glucose transporter (GLUT-4) in patients with common-type non-insulin-dependent diabetes mellitus, J Clin Invest. 1991; 88(4):1323-1330.
A. Regginato et al., Antidiabetic and hypolipidemic potential of Campomanesia xanthocarpa seed extract obtained by supercritical CO₂, Braz. J. Biol. 2020.
Costa et al., Morphoanatomical study, seasonal variation, and larvicidal activity of volatile oils from the leaves of Campomanesia pubescens (DC.) O. Berg (Myrtaceae), Research, Society and Development, v. 10, n. 3, e35610313412, 2021.

Amaral É. V. E. J. et al., Campomanesia adamantium and Campomanesia pubesncens are distinct species? Use of palynology and molecular markers in taxonomy, Rodriguésia 71: e02652018. 2020.

Oliveira et al., Chemical composition of essential oil extracted from leaves of Campomanesia adamantium subjected to different hydrodistillation times, Ciência Rural, v. 47, n. 1, 2017.

1. A pharmaceutical composition for controlling glycemic rate, comprising: Campomanesia pubescens essential oil.

2. The pharmaceutical composition according to claim 1, wherein the essential oil of Campomanesia pubescens is in a concentration of about 90 μl to about 500 μl.

3. The pharmaceutical composition according to claim 1, further comprising a pharmaceutically acceptable excipient.

4. The pharmaceutical composition according to claim 1, wherein said composition is administered orally.

5. The pharmaceutical composition according to claim 1, wherein said composition is in the form of a capsule.

6. A phytomedicine for controlling glycemic rate, comprising: capsules containing between 90 μl and 500 μl of the an essential oil obtained by extracting the leaves of Campomanesia pubescens plants.

7. A kit for controlling glycemic rate, comprising: (a) Campomanesia pubescens essential oil in a dosage ranging from about 90 μl to about 500 μl; and (b) instructions for use.

8. A process for preparing the pharmaceutical composition according to claim 1, said process comprising the steps of:
(a) collecting leaves of Campomanesia pubescens in a period before the fruit ripens;
(b) cleaning with water;
(c) extracting the essential oil by at least one of hydrodistillation or steam drag; and
(d) after extracting the oil, keeping in an amber glass to preserve its composition until final encapsulation.

9. A use of the pharmaceutical composition according to claim 1, wherein said use is for the manufacture of a pharmaceutical composition for controlling glycemic rate in an individual in need thereof.

10. A use of the pharmaceutical composition according to claim 1, wherein said use is for the manufacture of a phytomedicine for controlling glycemic rate in an individual in need thereof.

11. A use of the pharmaceutical composition according to claim 1, wherein said use is for the manufacture of a drug to at least one of treat or prevent diabetes.
